(19) United States
(12) Patent Application Publication
(10) Pub. No.: US 2015/0306355 A1
(43) Pub. Date: Oct. 29, 2015

(54) Guidewires with variable rigidity

(71) Applicant: Mark Edman Idstrom, Kansas City, MO (US)

(72) Inventor: Mark Edman Idstrom, Kansas City, MO (US)

(21) Appl. No.: 14/662,180

(22) Filed: Mar. 18, 2015

Related U.S. Application Data
(63) Continuation-in-part of application No. 14/263,674, filed on Apr. 28, 2014.
(60) Provisional application No. 62/102,561, filed on Jan. 12, 2015.

(51) Int. Cl. A61M 25/09

(52) U.S. Cl. CPC A61M 25/09; A61M 2025/09008; A61M 2025/09075; A61M 2025/0915

(57) ABSTRACT

Guidewires are disclosed being configured to achieve variable rigidity to facilitate percutaneous exploration and the traversal of overlying elements such as catheters, for instance in performing the Seldinger technique. Embodiments of guidewires comprise sheaths and cores configured to be tightened together to achieve stiffness of the guidewire. Embodiments further include cores having an axial passage, along with one or more sections capable of independent activation to achieve variable rigidity. Embodiments also include guidewires comprising combinations of solid, wound metallic, polymeric coils, or woven meshes, with attached or detached polymer coatings. Cores may be configured to increase or decrease pressure, pass acoustic, ultrasonic or other mechanical energy, pass one or more metallic cores to interact with the distal mechanical elements, or as a channel to pass wires to activate the distal materials in the sections and distal tip.

### CROSS-REFERENCE TO RELATED APPLICATION

This application is a continuation-in-part of U.S. patent application Ser. No. 14/263,674 filed on Apr. 28, 2014, and also claims priority to U.S. provisional patent application No. 62/102,561 filed on Jan. 12, 2015.

### BACKGROUND

Minimally invasive surgical techniques are an important aspect of medical procedures. Such procedures often require access to blood vessels, structures, organs, or cavities from small apertures at a distance. For example, in certain angioplasty procedures, from a small incision in the wrist or groin, a catheter may be advanced through the cardiovascular to a blocked or restricted artery. A balloon attached to the catheter, when positioned within the blockage, may then be inflated to radially expand against the restriction to enlarge the opening and increase the blood flow. Balloon catheters include over-the-wire designs requiring little support or control, allowing the placement of a small steerable wire through the restriction facilitating the catheter which can track the wire across the blockage. To reach areas of blood vessels restriction, guidewires often must traverse shallow or sharp turns, circuitous paths, pass competing branches, and cross disease and/or narrowed vessels. This may be accomplished by an operator advancing and withdrawing a guidewire while rotating a pre-formed tip into a favorable position while observing via fluoroscopy. As the guidewire advances deeper into the vessels in smaller and more diseased segments, increased resistance occurs between the guidewire and the blood vessel walls.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments include guidewires having variable rigidity. In certain embodiments, variable rigidity may be achieved via cores comprising solid wires or walled tubes. Cores may include coatings and may be stiffer than other sections of the guidewire, and function to guide and support a catheter (or other element) and transmit action and support rotation from an operator to the distal end. Intermediate sections may be interspersed axially and be more or less flexible than the core. In certain embodiments, variable rigidity may be achieved via a sheath disposed around a core. In one example, a sheath may comprise a coil or spring surrounding a core. Intermediate sections may transmit motion and rotation in addition to guiding an overlying catheter (or other element). Flexibility may be complimentary to permit the guidewire to conform to the curvature and tortuosity of the vasculature. A distal tip may connect via a distal joint, and may additionally include a curve allowing the guidewire to be directed from an operator.

### BRIEF DESCRIPTION OF THE DRAWINGS

FIG. 1A shows a cross sectional view of an exemplary embodiment of the present invention;

FIG. 1B shows another cross sectional view of an exemplary embodiment of the present invention;

FIG. 2A depicts a cross sectional view of an exemplary embodiment of the present invention;

FIG. 2B depicts another cross sectional view of an exemplary embodiment of the present invention;

FIG. 3 depicts still another cross sectional view of an exemplary embodiment of the present invention;

FIG. 4 illustrates still another cross sectional view of an exemplary embodiment of the present invention;

FIG. 5A illustrates a cross sectional view of an exemplary embodiment of the present invention;

FIG. 5B illustrates a cross sectional view of an exemplary embodiment of the present invention;

FIG. 6A depicts a cross sectional view of an exemplary embodiment of the present invention;

FIG. 6B depicts a cross sectional view of an exemplary embodiment of the present invention;

FIG. 7A shows another cross sectional view of an exemplary embodiment of the present invention;

FIG. 7B illustrates another cross sectional view of an exemplary embodiment of the present invention;

FIG. 7C illustrates still another cross sectional view of an exemplary embodiment of the present invention;

FIG. 8 shows a cross sectional view of an exemplary embodiment of the present invention;

FIG. 9 depicts another cross sectional view of an exemplary embodiment of the present invention;

FIG. 10A illustrates a cross sectional view of an exemplary embodiment of the present invention;

FIG. 10B illustrates another cross sectional view of an exemplary embodiment of the present invention;

FIG. 11 depicts a cross sectional view of an exemplary embodiment of the present invention;

FIG. 12A shows a longitudinal cross-sectional view of an exemplary embodiment of the present invention;

FIG. 12B shows a transverse cross-sectional view of an exemplary embodiment of the present invention;

FIG. 12C shows another transverse cross-sectional view of an exemplary embodiment of the present invention;

FIG. 12D shows another transverse cross-sectional view of an exemplary embodiment of the present invention;

FIG. 13A depicts a transverse cross-sectional view of an exemplary embodiment of the present invention;

FIG. 13B depicts a transverse cross-sectional view of yet another exemplary embodiment of the present invention;

FIG. 13C depicts a transverse cross-sectional view of yet another exemplary embodiment of the present invention;

FIG. 13D depicts a transverse cross-sectional view of yet another exemplary embodiment of the present invention;

FIG. 13E depicts a transverse cross-sectional view of yet another exemplary embodiment of the present invention;

FIG. 14A illustrates a longitudinal cross-sectional view of an exemplary embodiment of the present invention;

FIG. 14B illustrates another longitudinal cross-sectional view of an exemplary embodiment of the present invention;

FIG. 14C illustrates yet another longitudinal cross-sectional view of an exemplary embodiment of the present invention;

FIG. 14D illustrates yet another longitudinal cross-sectional view of an exemplary embodiment of the present invention;

FIG. 15A shows a longitudinal cross-sectional view of an exemplary embodiment of the present invention;

FIG. 15B shows a transverse cross-sectional view of an exemplary embodiment of the present invention;

FIG. 15C shows another longitudinal cross-sectional view of an exemplary embodiment of the present invention;

FIG. 15D shows another longitudinal cross-sectional view of an exemplary embodiment of the present invention;

FIG. 15E shows another longitudinal cross-sectional view of an exemplary embodiment of the present invention;

FIG. 16A depicts a longitudinal cross-sectional view of an exemplary embodiment of the present invention;

FIG. 16B depicts a transverse cross-sectional view of an exemplary embodiment of the present invention;

FIG. 16C depicts another longitudinal cross-sectional view of an exemplary embodiment of the present invention;

FIG. 16D depicts another longitudinal cross-sectional view of an exemplary embodiment of the present invention;

FIG. 16E depicts still another longitudinal cross-sectional view of an exemplary embodiment of the present invention;

FIG. 17A shows a longitudinal cross-sectional view of an exemplary embodiment of the present invention;

FIG. 17B shows a transverse cross-sectional view of an exemplary embodiment of the present invention;

FIG. 17C shows another longitudinal cross-sectional view of an exemplary embodiment of the present invention;

FIG. 17D shows another longitudinal cross-sectional view of an exemplary embodiment of the present invention;

FIG. 17E shows yet another longitudinal cross-sectional view of an exemplary embodiment of the present invention;

FIG. 18A illustrates a transverse cross-sectional view of an exemplary embodiment of the present invention;

FIG. 18B illustrates another transverse cross-sectional view of an exemplary embodiment of the present invention;

FIG. 18C illustrates another transverse cross-sectional view of an exemplary embodiment of the present invention;

FIG. 18D illustrates still another transverse cross-sectional view of an exemplary embodiment of the present invention;

FIG. 19A is a longitudinal cross-sectional view of an exemplary embodiment of the present invention;

FIG. 19B is a transverse cross-sectional view of an exemplary embodiment of the present invention;

FIG. 19C is another transverse cross-sectional view of an exemplary embodiment of the present invention;

FIG. 20A is a partial perspective view of an exemplary embodiment of the present invention;

FIG. 20B is another perspective view of an exemplary embodiment of the present invention;

FIG. 20C is a transverse cross-sectional view of an exemplary embodiment of the present invention;

FIG. 20D is another perspective view of an exemplary embodiment of the present invention;

FIG. 21A is a partial perspective view of an exemplary embodiment of the present invention;

FIG. 21B is a perspective view of an exemplary embodiment of the present invention;

FIG. 21C is a transverse cross-sectional view of an exemplary embodiment of the present invention;

FIG. 21D is another perspective view of an exemplary embodiment of the present invention;

FIG. 22A is a perspective sectional view of an exemplary embodiment of the present invention;

FIG. 22B is a transverse cross-sectional view of an exemplary embodiment of the present invention;

FIG. 22C is another perspective view of an exemplary embodiment of the present invention;

FIG. 23A is a transverse cross-sectional view of an exemplary embodiment of the present invention;

FIG. 23B is a partial perspective view of an exemplary embodiment of the present invention;

FIG. 23C is a cutaway perspective view of an exemplary embodiment of the present invention;

FIG. 23C is a cutaway perspective view of an exemplary embodiment of the present invention; and

FIG. 23D is a cutaway perspective view of an exemplary embodiment of the present invention.

### DETAILED DESCRIPTION

The subject matter of the present invention is described with specificity to meet statutory requirements. The description itself is not intended to limit the scope of this patent, however. Rather, the inventor has contemplated that the claimed subject matter might also be embodied in other ways, including via different components or combinations of components similar to the ones described in this document, as well as embodiments comprising methods or systems, including embodiments in conjunction with other present or future technologies.

In general, the more tortuous and acutely angled the sections of vasculature to traverse, the softer the guidewire required to reach a target. However, stiffer guidewires may be advantageous for use with overlying elements (such as a catheter) because such guidewires allow the overlying elements to more efficiently follow and reach a target. For instance, a catheter may be prevented from reaching the target when used over a soft guidewire because the catheter may eject a soft guidewire from the vessel, or dig into the vessel at a sharp curve, rather than follow the guidewire. This may be addressed via serially exchanging from the initial soft wire, to a soft catheter, to a firmer guidewire, then a firmer catheter, and so on until an adequately firm supporting guidewire is in place. These serial exchanges are time consuming, can cause vessel injury, require extra supplies, can lead to contamination at the insertion site, and can stimulate spasm in the vessel ultimately leading to the failure of the procedure. Stiff guidewires may have disadvantages in taking tortuous anatomy and forcing it to conform to the relatively straight shape of the guidewire, which may stimulate spasm of the vessel, distort the anatomy (which changes the location of the target), and increase the risk of perforation and vessel injury.

Embodiments of the present invention include guidewires facilitating the maintenance of control and flexibility for access to targets (such as percutaneous medical targets), and which also may be selectively stiffened to support and facilitate the guidance of an element (such as a catheter) to the target.

In one embodiment in accordance with the present invention, a guidewire is disclosed comprising a core, and a sheath disposed around the core. The sheath is configured to tighten to the core. As used herein, when elements are described as tightening or being tightened to another element, it is contemplated that in some instances, only portions of the elements may be tightened together, rather than the entire elements. It is also contemplated that entire elements may be tightened together. Both such configurations are contemplated as being within the scope of the present invention. Further, the guidewire may comprise a means for tightening the sheath to the core. Additionally, the core may comprise a coil. The guidewire may by configured to operatively stiffen via a suction element configured to retractably tighten the sheath to the core. As used herein, when guidewires are described as “stiffening,” being “stiffened,” or becoming “stiff,” etc., it is contemplated that only portions of the guidewire may become stiffened (rather than the entire guidewire). It is also contemplated that the entire guidewire may be stiffened in configurations. Such embodiments are within the bounds contemplated by the inventor. Keeping with the previous example, additionally or alternatively, the sheath may comprise a helical braid, which may be tightened to the core via the application of an axially retracting force. Still in addition or in the alternative, the sheath may comprise an outer coil, which may be tightened to the core via the application of a force, such as a rotational or retracting force. These, or other configurations or means, may tighten the sheath to the core, stiffening the guidewire.

In another embodiment in accordance with the present invention, a guidewire is disclosed. The guidewire includes a core. The guidewire further includes a sheath disposed around the core. In embodiments, the core may be configured to be tightened to the sheath. In certain embodiments, the guidewire includes a means for tightening the core to the sheath. For instance, the core of the guidewire may comprise a coil, and the coil may be twisted to tighten it to the sheath, which may stiffen the guidewire. The guidewire may additionally include a tip having an inner surface, and the core may be fixed to the inner surface of the tip. This configuration allows the coil to be twistingly expanded when rotation is applied. The core may also comprises a helically wound braid, where the core is tightened to the sheath via the application of axial force to the helically wound braid.

In yet another embodiment in accordance with the present invention, a guidewire is disclosed that includes a core having a first end, a second end distal from the first end, an outer surface, and an intermediate section. The second end defines a tip, the tip being optionally configured to facilitate percutaneous exploration. The intermediate section may further be configured to increase in rigidity upon activation. The guidewire may also be configured to provide a pathway for an overlying element, such as a catheter. The outer surface may be selected from percutaneously compatible materials. The core may also comprise an electroactive material, such as an electroactive polymer. The electroactive material may be electrically stimulated to increase the rigidity of portions of the guidewire. Alternatively or additionally, the core may comprise a coil, and the intermediate section may be configured for activation via rotation of the coil. Still alternatively or additionally, the shaft may include a non-Newtonian fluid, and wherein the intermediate section is configured for activation via mechanical stimulation of the non-Newtonian fluid. One example of such a mechanical stimulation includes oscillatory stimulation.

In another aspect of embodiments, a guidewire comprise a core having an axial passage; an intermediate section comprising a coil coupled to a polymer membrane sheath; and a blunt tip coupled to the polymeric membrane and wound coil. The guidewire may be configured to cause the sheath and coil to interact and stiffen the guidewire. This may be accomplished via induced positive pressure in the axial passage, negative pressure in the axial passage, combinations of positive and negative pressures, or other means. It will be understood that various other means of achieving variable rigidity are within the bounds of the present inventions. As one example, another aspect of embodiments include add coating to the inner coil, or would add a wound coil to the outer membrane.

Another aspect of embodiments includes a guidewire comprising a core, the core being hollow, and wherein the inner portion of the core includes stiffening elements for selectively stiffening the guidewire. For instance, stiffening elements may be chosen from the group consisting of linear wires, metallic materials (e.g. Nitinol), magnetic metals, carbon fiber, polymeric fibers, metallic granules, or polymeric granules. As an example of means for stiffening the guidewire, a change from positive to negative pressure may create an interaction between the polymeric membrane and the central solids leading to a stiffening of the guidewire.

In still another aspect of embodiments, a guidewire includes a core, one or more intermediate sections, and a tip. The core (and intermediate sections in certain instances) may comprise a wound coil. The wound coil includes an inner portion, and may be configured to retain an inflatable element (such as a balloon) within the inner portion. Such an inflatable element, when inflated, may be configured to expand radially against the wound coil, stiffening the guidewire. When stiffened, the guidewire may advantageously provide a path for overlaying elements (such as a catheter, grapple, hook, optical unit, or other surgical element) to traverse. When necessary, the inflatable element may be deflated, reducing the stiffness of the guidewire, facilitating the further guidance or retraction of the guidewire. In alternate configurations, the inner portion of the wound coil may retain materials which, when activated (via mechanical, chemical, or electrical means), hardens, stiffens, or swells, stiffening the guidewire. In yet another aspect, the inner portion may retain additional coils, woven meshes, or other mechanical elements attached to the inner portion that may be expanded against inner portion when activated, stiffening the guidewire. The activation may be achieved by through motion or rotation on the wire or mesh. Depending on the configuration of the coil or mesh, the guidewire may be stiffened from proximal to distal, or distal to proximal.

In another aspect of embodiments, a guidewire includes a core containing two fibers located adjacent to one another, a sheath disposed around the core and a means for coupling the fibers, which serves to stiffen the guidewire. In embodiments, the sheath may be configured to retract upon the core coupling the fibers such that the fibers cannot slide independently of one another, stiffening the guidewire. For instance, the sheath may contain electroactive polymers that may be electrically stimulated to flex, retracting the sheath against the core, which couples the fibers and stiffens the guidewire. As another example, the guidewire may be coupled to a pump. When activated, the pump may extract fluid (such as water, blood, or air) from the interior, causing the sheath to retract against the core, which couples the fibers and stiffens the guidewire. As yet another example, the sheath may contain a flexible inner-wall and a rigid outer-wall separated by an interior channel that is connected to a pump. Activating the pump may increase the pressure in the sheath's interior channel, which causes the sheath's flexible inner-wall to retract against the core, coupling the fibers and stiffening the guidewire. In embodiments, the core may be configured to expand upon the sheath causing the fibers to couple (to other elements of the core, or to the sheath) and stiffening the guidewire. For example, the core may contain electroactive polymers that may be electrically stimulated to expand causing the fibers to couple, tightening the core to the sheath and stiffening the guidewire. As another example, the core may include a balloon connected to a pump. Activating the pump may inflate the balloon causing the fibers to couple and tightening the core to the sheath. The core may also include a coil, which may expand against the fibers when twisted. Expanding the coil may couple the fibers, tighten the core against the sheath and stiffen the guidewire. In another aspect of embodiments, the fibers may be configured to “stick” together during activation of the guidewire. For instance, the fibers may be coated or constructed from a material with adhesive properties. The fibers may also be textured to promote mechanical adhesion.

Another aspect of embodiments includes a core having two or more fibers arranged in a prescribed pattern, and a sheath disposed around the core. Upon activation, the fibers assume a second prescribed pattern that serves as scaffolding for the sheath as the guidewire is stiffened. In embodiments, the second prescribed pattern may minimize cross-sectional distortion of the guidewire as the guidewire is stiffened. For example, the core may comprise a packing of seven similarly-sized cylindrical fibers wherein six fibers are disposed around a seventh fiber. When the guidewire is activated, by creating a negative pressure differential in the guidewire's interior for example, these seven fibers will assume a pattern of tangency that is symmetrical and minimizes cross-sectional distortion of the sheath as the sheath retracts upon the core. Alternate configurations of fibers that preserve the guidewire's shape prior to stiffening are possible, and may include multiple layers of fibers disposed around a central fiber, fibers of different shapes and fibers of different sizes.

Another aspect of embodiments includes a core and a sheath, the sheath adapted to retract upon the core. The sheath comprises a first section, a second section, and a joining section coupling the first section and the second section. The joining section is deformable relative to the first section and the second section, allowing for the controlled folding of the sheath at the joining section as the guidewire is activated (for instance, by tightening the sheath to the core). For example, the joining section may comprise a flexible material that folds during activation. As another example, the joining section may be creased such that the sheath folds along the crease during activation. Finally the joining material may be scored such that the sheath folds along the scoring during activation. In certain embodiments, the core may include two cylindrical fibers that are loosely coupled to the sheath such that the joining sections align with a point mid-way between the fibers. When the guidewire is activated, folding may preferentially occur at the joining sections in between the fibers where it is less likely to interfere with passage of an external element over the guidewire.

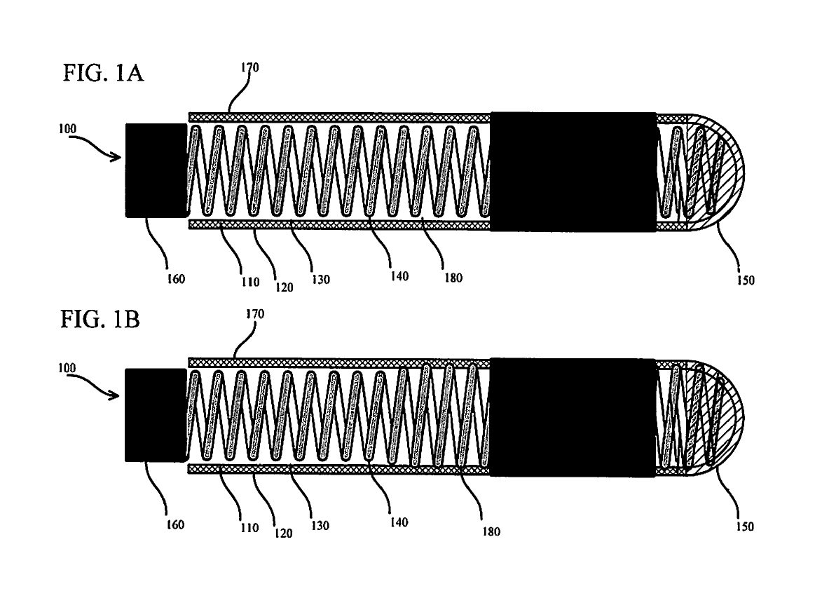

Having briefly described an overview of embodiments of the present invention, exemplary guidewires are described. Referring to FIG. 1A, a cross sectional view of guidewire 100 is shown. Guidewire 100 includes sheath 110, which has outer surface 120 and inner surface 130. Guidewire 100 further includes first end 170 and second end 150, first end 170 being distal from second end 150. In this instance, second end 150 defines a tip. Guidewire 100 further includes core 180, the core comprising coil 140 and shaft 160. Coil 140 and shaft 160 are coupled together. In this instance, shaft 160 and coil 140 comprise a means for tightening to sheath 110 via rotational force applied to shaft 160. Guidewire 100 is configured such that coil 100 may be tightened via the application of rotational force via shaft element 160. Referring now to FIG. 1B, another cross sectional view of guidewire 100 is illustrated. In this instance, coil 140 is shown having been rotationally expanded so as to tighten to sheath 110. (As can be seen, guidewire 100 may be stiffened along portions, rather than entire length of guidewire 100.) Guidewire 100 operatively stiffens when coil 140 is tightened against sheath 110, facilitating the guidance of an overlying element toward a target. For instance, as with the guidance of a medical element—such as a catheter—in performing the Seldinger technique, or other techniques.

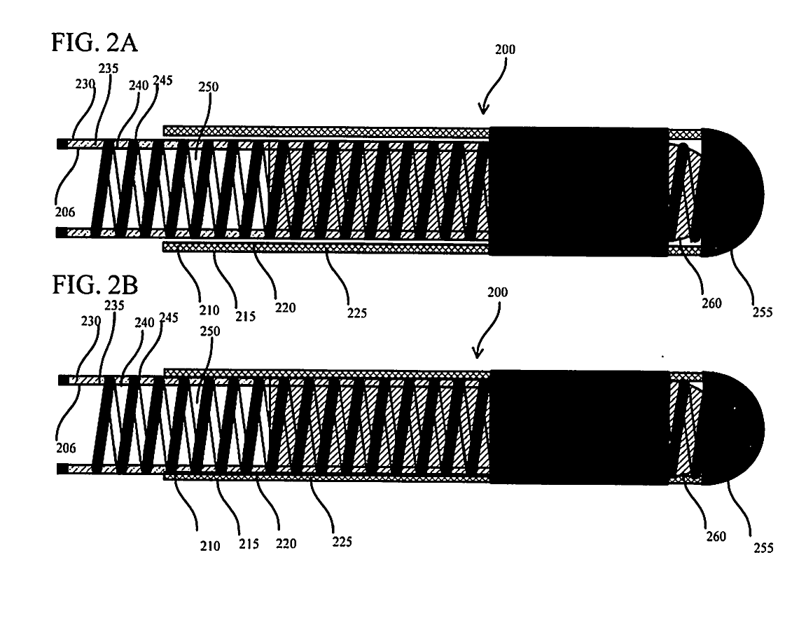

Referring now to FIG. 2A, a cross sectional view of a guidewire 200 is shown. Guidewire 200 includes sheath 210, which has outer surface 215, inner surface 220, and inner portion 225. In some embodiments, inner portion 225 includes an electrically conductive material or structures. Guidewire 200 further includes core 230, which comprises covering 235 having an inner surface 240 and outer surface 245. Inner surface 245 defines an interior, which houses coil 250, which in some embodiments is electrically conductive. At the distal end of guidewire 200 is tip 255, and, in the particular configuration depicted, tip 260 of core 230. The boundary between sheath 210 and core 230 define an interface. Various means may be employed to tighten sheath 210 to core 230 (wholly or in portions). For instance, suction may be applied to the interface, drawing sheath 210 toward core 230, stiffening guidewire 200. Another means includes applying rotational force to coil 250, which serves to expand coil 250 along with core 230 until outer surface 245 contacts inner surface 220, stiffening guidewire 200. Yet another means includes providing a positive pressure to the cavity defined by inner surface 240, inflatably expanding core 230 to tighten to sheath 210. Still another means, in the case where core 230 includes electrically conductive material and sheath 210 comprises electrically conductive material, includes inducing attractive magnetic forces between core 230 and sheath 210. For instance, differing electrical currents may be applied to core 230 and sheath 210, inducing attractive magnetic fields such that sheath 210 and core 230 tighten together, stiffening guidewire 200. Various other mechanical, electrical, magnetic, and chemical means for tightening sheath 210 and core 230 together may be apparent to those of skill in the art, and are contemplated as within the bounds of the present invention. FIG. 2B depicts guidewire 200 wherein sheath 210 and core 230 are tightened together. Additionally, configurations include selectively applying tightening means to portions of sheath 210 and core 230, operatively stiffening intermediate sections of guidewire 200. For instance, guidewire 200 may include multiple intermediate sections, each defined by separate activation sections. For instance, activation sections may comprise different circuit paths electrically accessible from a proximal end of guidewire 200. When a current is applied to a particular circuit path, the intermediate section defined by that circuit path may be activated, stiffening that intermediate section of the guidewire.

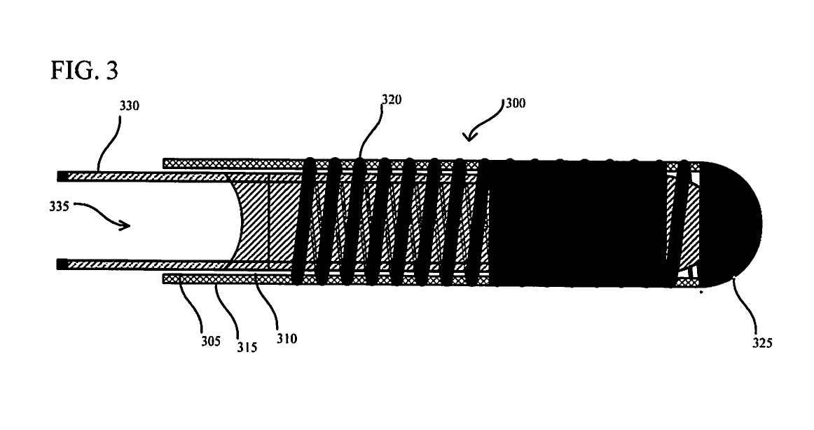

FIG. 3 will now be discussed, which depicts a cross sectional view of guidewire 300. Guidewire 300 includes sheath 305, which comprises inner surface 310, outer surface 315, and tip 325. Tip 325 may be fashioned to provide a blunt end for guidewire 300, which may be advantageous for percutaneous insertion and exploration of intravenous, intra-arterial, or intra-cavity exploration to reach a medical target Inner surface 310 defines an interior, which houses core 330. In the depicted embodiment, sheath 305 is coupled to coil 320, which provides structure and a configuration for tightening sheath 305 to core 330 (for instance by applying rotational force to coil 320 to stiffen guidewire 300). Core 330 defines interior 335. In some embodiments, core 330 comprises an inflatable element, which comprise a means for tightening core 330 to sheath 305. Means for tightening core 330 to sheath 305 further include applying positive pressure to interior 335, (such as by expanding core 330 such as to tighten to sheath 305). Outer surface 315 may comprise a variety of materials, or coatings, advantageous for percutaneous insertion.

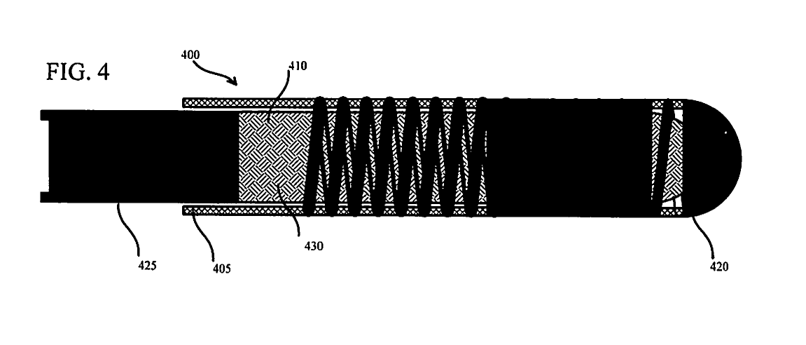

Turning now to FIG. 4, a cross sectional view of guidewire 400 is depicted. Guidewire 400 comprises sheath 405, which includes tip 420. Sheath 405 defines an interior, which houses core 410. In the embodiment depicted, core 410 comprises a helical braid, such as a biaxial braid (as seen in, for example, Chinese finger traps). Core 410 further comprises pusher 425 and intermediate section 430. An axial force may be applied to pusher 425, radially expanding core 410 due to the biaxial weave composition, tightening core 410 against sheath 405, increasing the rigidity of guidewire 400. Additional means for tightening sheath 405 to core 410 are contemplated by the inventor, as well as additional means for tightening core 410 to sheath 405—such as differing configurations of pushers, coils, sheaths, cores, and combinations thereof.

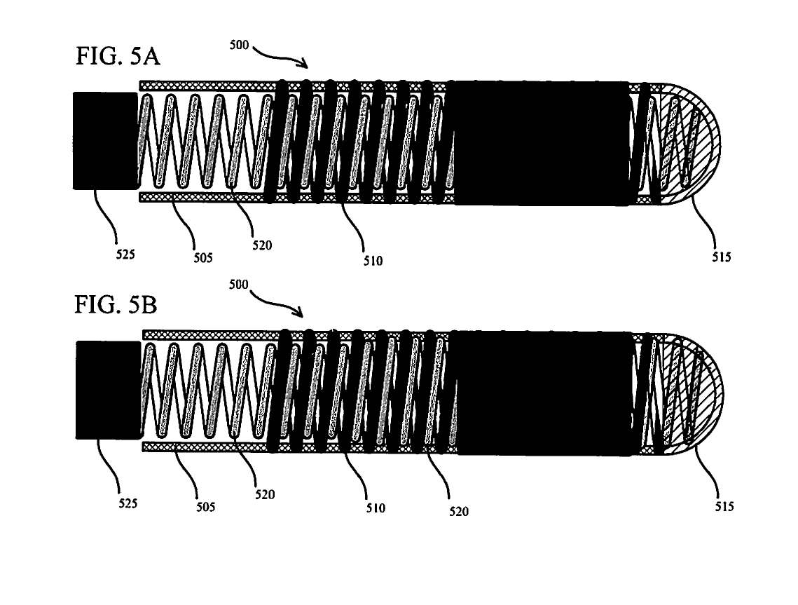

Moving on to FIG. 5A, a cross sectional view depicts another aspect in accordance with embodiments. Guidewire 500 comprises sheath 505 having tip 515. Sheath 505 defines a interior and is coupled to coil 510. Core 520 comprises a coil, which is operatively interlinked with coil 510. Core 520 is coupled to rotational element 525, which is configured to facilitate the application of rotational force to coil 520, activating guidewire 500 to increase rigidity. Means for tightening core 520 to sheath 505 include the coil composition of core 520 and coil 510, as well as rotational element 525 (though the latter may or may not be necessary, or may comprise various alternative configurations). FIG. 5B illustrates an embodiment wherein core 520 is tightened to sheath 505, also illustrating that a distal first section may be tightened independently of a proximal second section.

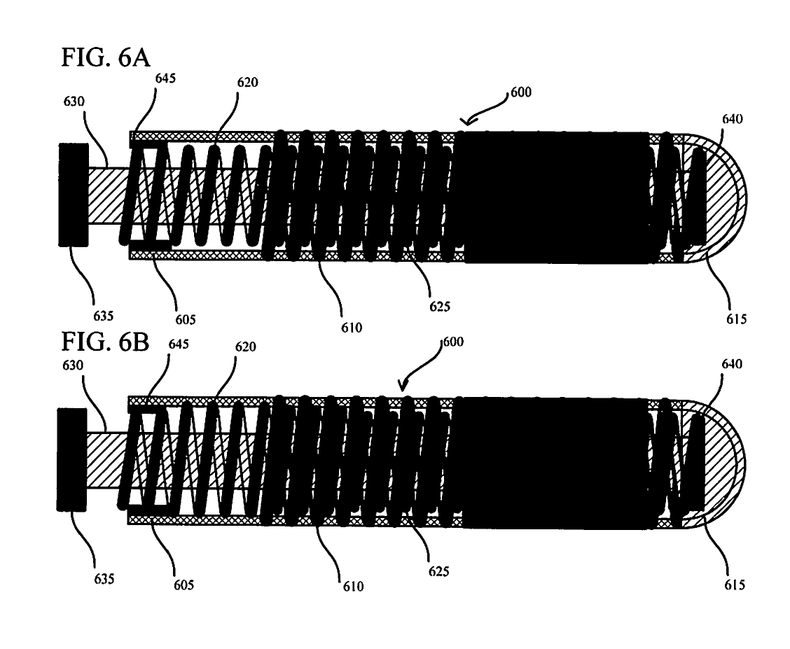

Turning now to FIG. 6A, a cross sectional view of guidewire 600 is depicted. Guidewire 600 comprises sheath 605, which defines distal tip 615. Distal tip 615 may comprise a soft material formed into a blunt shape, preventing puncture as guidewire 600 is operated for percutaneous exploration. Sheath 605 further comprises coil 610, which is configured to provide structure to sheath 605, as well as to allow sheath 605 to be operatively tightened to core 620. Core 620 comprises coil 625, which is interlinked with coil 610. Core 620 further comprises shaft 630, which is coupled at portion 640 to coil 625, and coil 625 is further coupled to sheath 605 at end 645. This configuration advantageously allows rotational force on shaft 630 to be translated to coil 625 near end 645, radially expanding core 620 to tighten to sheath 605 near end 645, operatively guided via coil 610. Grip 635 provides traction in order to apply rotational force to coil 625 via shaft 630. Coil 625 thus comprises a means for tightening core 620 to sheath 605, operatively stiffening guidewire 600. FIG. 6B depicts guidewire 600 wherein a proximal first section is tightened independently of a distal second section.

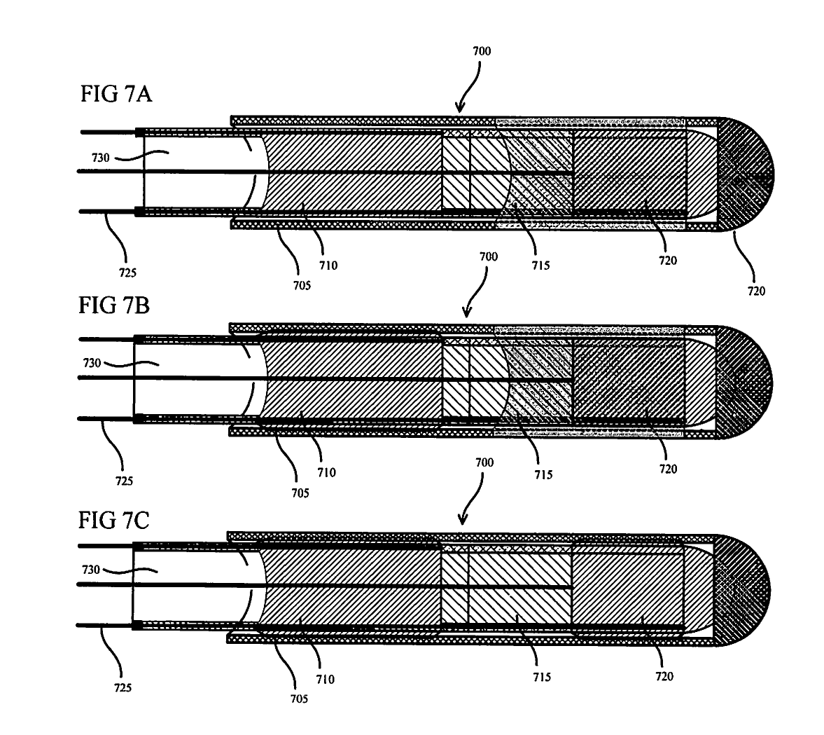

FIG. 7A illustrates a cross sectional view of guidewire 700, which illustrates a configuration comprising multiple intermediate sections, each capable of being independently stiffened. Guidewire 700 comprises core 725, which comprises an electrically conductive cylindrical structure that may be magnetically activated via electrical stimulation. Guidewire 700 further comprises sheath 705. Core 725 further comprises first intermediate section 710, second intermediate section 715, and third intermediate section 720. Each of these intermediate sections is electrically coupled to end 730 via independent circuits, such that each intermediate section is capable of being electrically stimulated independently of other intermediate sections. For instance, electrical stimulation may be applied to the circuit path coupled to second intermediate section 715 independently of first intermediate section 710 and third intermediate section 720, inducing a magnetic activation to attract second intermediate section 715 to sheath 705. In such a case, only second intermediate section 715 is electrically activated, while first intermediate section 710 and third intermediate section 720 remain non-activated. If desired, because the intermediate sections are independently capable of activation, first intermediate section 710 and third intermediate section 720 may also be electrically activated, stiffening those sections in addition or in the alternative to second intermediate section 715. For illustration, FIG. 7B depicts a further cross sectional view of guidewire 700 wherein first intermediate section 710 is magnetically activated via electrical stimulation, tightening first intermediate section 710 to sheath 705, stiffening guidewire 700 around first intermediate section 710. As can be seen, second intermediate section 715 and third intermediate section 720 are not magnetically activated in FIG. 7B, and as such, these intermediate sections are not tightened to sheath 705. As another illustration, FIG. 7C depicts a further scenario wherein third intermediate section 720 is additionally electrically stimulated, magnetically activating third intermediate section 720 and tightening it to sheath 705 as well as first intermediate section 710 (while second intermediate section 715 remains un-activated). Thus, it can be seen that each intermediate section may be independently activated, tightening independent sections of guidewire 700 as desired. As will be apparent, any number of intermediate sections may be activated in various combinations, achieving stiffness of portions of guidewire 700 according to desires. These and other configurations and means for tightening core 725 to sheath 705 are within the bounds of the invention, including having intermediate sections of sheath 705 capable of independent activation to be tightened to core 725. Still another means for said tightening involves intermediate sections independently coupled to end 730 via separate coil partitions, each intermediate section configured for activation via independent applications of mechanical or electrical force.

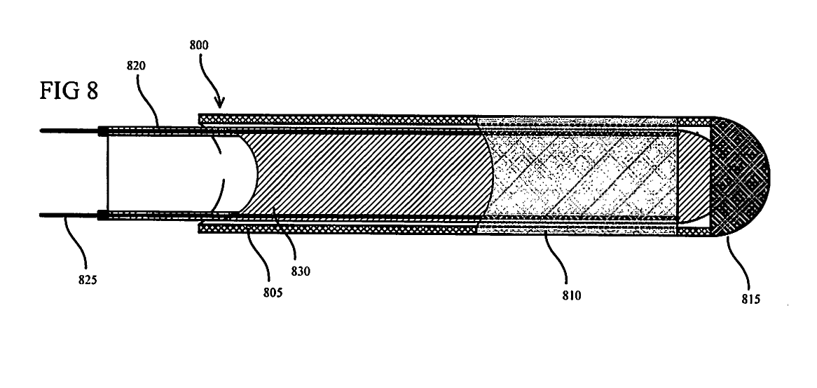

Turning now to FIG. 8, cross sectional view of guidewire 800 is depicted. Guidewire 800 comprises sheath 805, which may comprises coil 810. In this instance, coil 810 provides structure to a portion of sheath 805, but other elements may be operational to provide structure to sheath 805, including the material from which sheath 805 is constructed. For instance, sheath 805 may be constructed from polymer materials providing appropriate stiffness and flexibility. Sheath 805 further comprises tip 815, which comprises a soft material appropriate for percutaneous insertion and exploration. Guidewire 800 further comprises core 820, which comprises electrode 825, which traverses core 820 and may advantageously follow a path covering an appropriate cross-sectional area of core 820. Core 820 further comprises an interior, which houses electroactive material 830. Electroactive material 830 may be electrically stimulated via electrical activation of electrode 825. Various electroactive materials may be known to those of skill in the art, and include, for instance, electrically activated gels, piezoelectric materials in various structural configurations, or carbon nanotube materials.

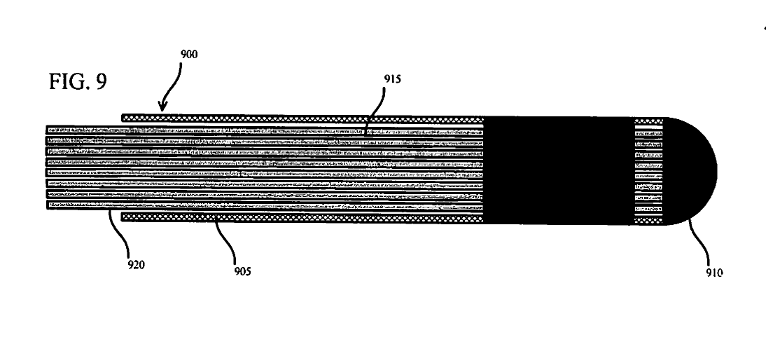

FIG. 9 depicts a cross sectional view of guidewire 900, which comprises sheath 905. Sheath 905 defines tip 910, which is configured for percutaneous exploration. Guidewire 900 further comprises core 915, which in guidewire 900 comprises multiple, flexible fibers operational to provide structure to guidewire 900 when used for medical or other techniques. The flexible fibers comprising core 915 may be structured in various manners, such as braided, threaded, twisted, or straightened. Interior space 920 may be operative to have a sectional force applied, tightening sheath 905 to core 915, operatively stiffening guidewire 900, or alternatively tightening core 915 to sheath 905.

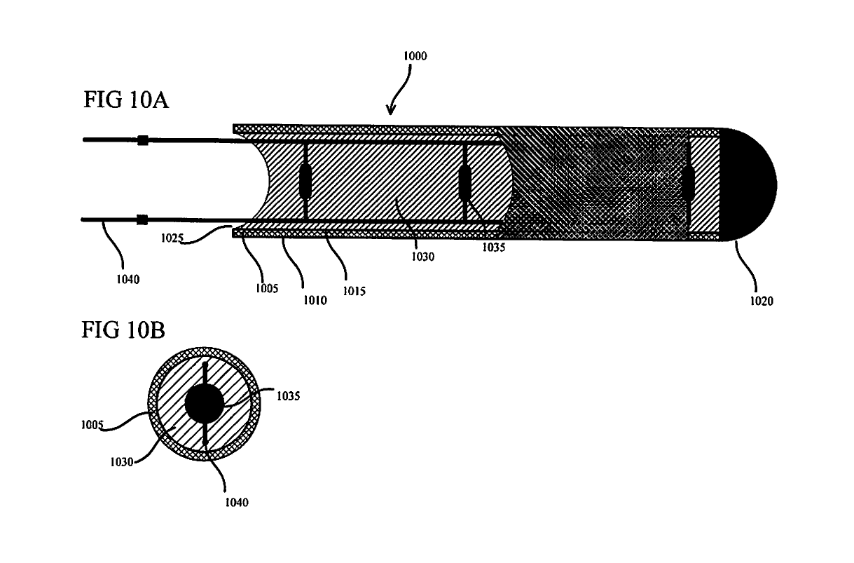

Turning now to FIG. 10A, cross sectional view of guidewire 1000 is depicted. Guidewire 1000 comprises sheath 1005, which comprises outer surface 1010, inner surface 1015, and tip 1020. Sheath 1005 defines interior 1025, which houses core 1030. Guidewire 1000 is configured to be stiffened via activation of core 1030. For instance, core 1030 may comprise a material activated via mechanical stimulation, such as a non-Newtonian fluid. Such a material could be activated, for example, via oscillatory stimulation, stiffening guidewire 1000 while such mechanical stimulation is applied. As another example, core 1030 may comprise an electroactive material, such as an electroactive polymer or piezoelectric material, which may be activated via electrical stimulation, for instance from electrodes embedded in sheath 1005, coupled to inner surface 1015, or housed within interior 1025 (as illustrated via electrode 1040). Upon electrical stimulation, electroactive material comprising core 1030 is activated, operatively stiffening guidewire 1000. As another example, core 1030 may comprise intertwined coils activated via rotational force, or may comprise a biaxial braid activated via axial force, either of which, when activated, stiffen guidewire 1000. Other functional configurations will be apparent, and include combinations of the above, interspersed according to sections for stiffening appropriate portions of guidewire 1000. FIG. 10B depicts a transverse cross section of guidewire 1000, illustrating that the interior of sheath 1005 is filled with core 1030.

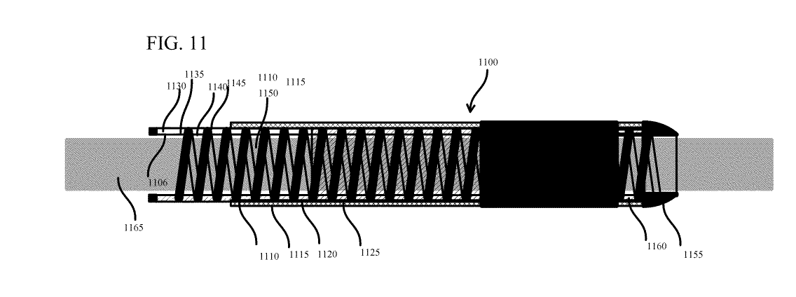

FIG. 11 depicts guidewire 1100 wherein sheath 1110 and core 1130 are tightened together. In this embodiment, tip 1155 is open allowing the hollow core 1106 to contain device 1165 which passes through the guidewire 1100. In some embodiments, the tightening of sheath 1110 and core 1130 may additionally tighten to device 1165. In other embodiments, device 1165 would remain free to move with the core 1106.

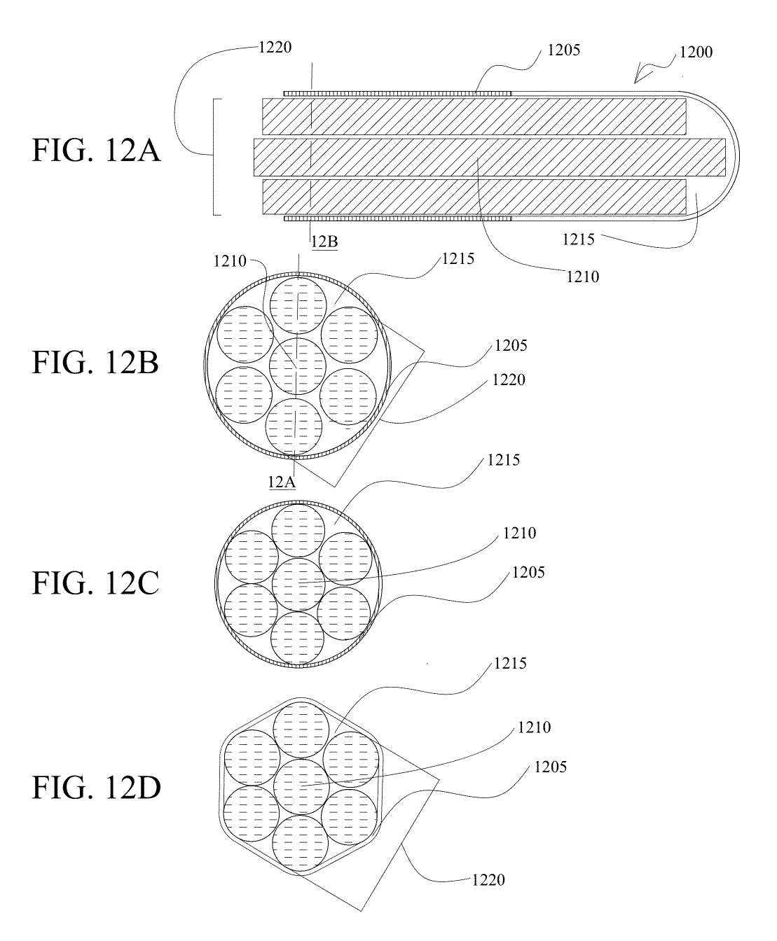

Turning now to FIGS. 12A, 12B, 12C and 12D, another embodiment is depicted. As shown, guidewire 1200 includes core 1220 having an arrangement of fibers that minimizes cross-sectional distortion of guidewire 1200 as guidewire 1200 is stiffened through a change in pressure inside sheath 1205. FIG. 12A shows a cross-sectional view of guidewire 1200 along its longitudinal axis. Guidewire 1200 comprises sheath 1205, which defines interior 1215. Interior 1215 houses core 1220, which comprises multiple, flexible, parallel fibers. FIG. 12B shows a cross section of guidewire 1200 in its flexible state. As can be seen in FIG. 12B, sheath 1205 is loosely coupled to core 1220, providing for flexibility of guidewire 1200. Core 1220 comprises 7 similarly- or identically-sized fibers that are arranged within interior 1215 such that six of these fibers are disposed around central fiber 1210. Interior 1215 is sufficiently large to allow the fibers to move longitudinally relative to one another but sufficiently small to preserve the lateral configuration of fibers within sheath 1205 as guidewire 1200 is utilized. Interior 1215 is configured to maintain a pressure differential relative to its environment, which may be manipulated during the use of guidewire 1200. For instance, interior 1215 may have a positive or neutral pressure differential, keeping sheath 1205 loosely coupled to core 1220, which renders guidewire 1200 relatively flexible (due to the ability of the fibers to move relative to each other and to the sheath). Guidewire 1200 may then be activated by causing a negative pressure differential in interior 1215, causing sheath 1205 to retract and conform to core 1220 (squeezing the fibers together so that they cannot move as easily relative to each other or to the sheath). FIG. 12C shows a transverse cross section of guidewire 1200 in its stiffened state. Interior 1215 may exhibit a negative pressure, causing the fibers within core 1220 to assume a pattern of tangency and causing core 1220 to interact with sheath 1205 in a manner that preserves the shape that guidewire 1200 had assumed prior to stiffening. FIG. 12D shows a transverse cross section of guidewire 1200 in a stage of advanced stiffening. Guidewire 1200 may be further activated through increased negative pressure within interior 1215 causing sheath 1205 to interact with the core 1220 more closely along the perimeter of core 1220.

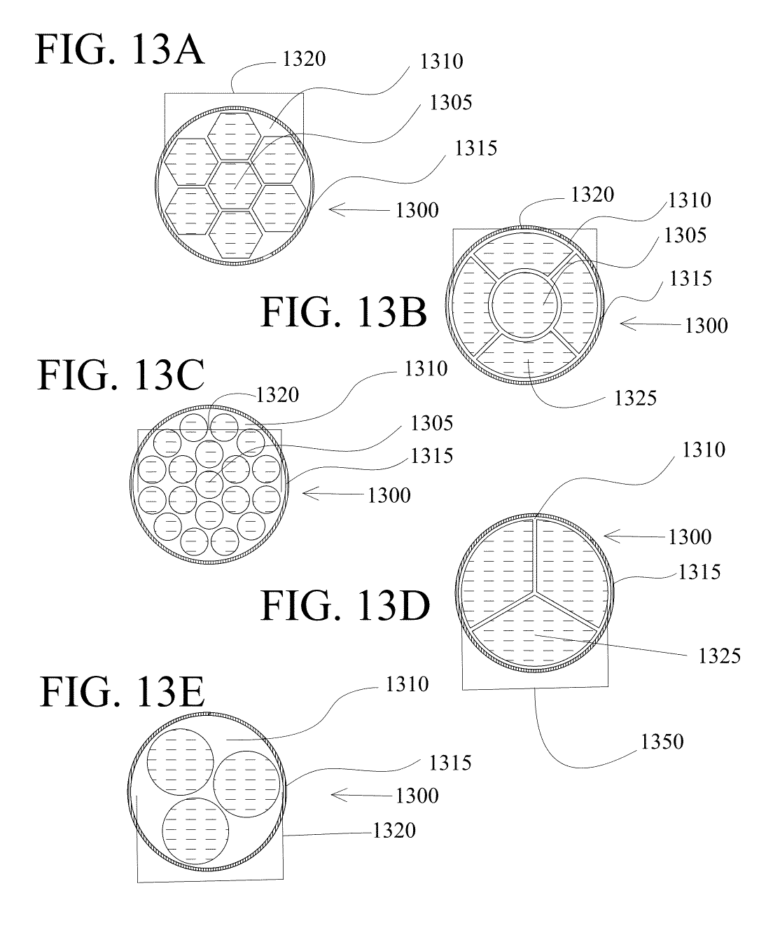

Depicted in 13A, 13B, 13C, 13D and 13E are transverse cross-sectional views of various guidewires in accordance with embodiments. These guidewires include cores having arrangements of multiple fibers that minimize cross-sectional distortion of the guidewires as the guidewires are stiffened. FIG. 13A includes six identically- or similarly-sized hexagonal fibers disposed around central fiber 1305 (together the fibers comprise core 1320). Guidewire 1300 may be activated by causing core 1320 to interact with sheath 1315, which causes the fibers within interior 1310 to tessellate in a manner that preserves the shape that guidewire 1300 had prior to stiffening. Other arrangements of non-cylindrical fibers which interface in a manner that preserves guidewire 1300's shape are possible and contemplated as within the scope of the present invention.

FIG. 13B depicts four elbow-shaped fibers (including fiber 1325) disposed around cylindrical fiber 1305. These four elbow-shaped fibers and cylindrical fiber 1305 comprise core 1320. Guidewire 1300 may be activated, causing core 1320 to interact with sheath 1315 in a manner that preserves the shape that guidewire 1300 had assumed prior to stiffening. Other arrangements involving a combination of fibers of various shapes which interface in a manner that preserves the guidewire's shape are possible and contemplated as within the scope of this invention.

FIG. 13C depicts another potential arrangement of fibers. In particular, FIG. 13C shows 18 identically- or similarly-sized cylindrical fibers arranged in two-coaxial layers disposed around central fiber 1305 (together comprising core 1320). Guidewire 1300 may be activated by causing a negative pressure differential in interior 1310, causing the fibers of core 1320 to assume a pattern of tangency and causing core 1320 to interact with sheath 1315 in a manner that preserves the shape that guidewire 1300 had assumed prior to stiffening. Other arrangements having multiple layers of fibers which interface in a manner that preserves the guidewire's shape are possible and within the scope of this invention.

FIG. 13D depicts three wedge-shaped fibers (including fiber 1325) within interior 1310 (the fibers comprising core 1320). Each fiber has a rounded edge that is loosely coupled to sheath 1315. Guidewire 1300 may be activated causing the fibers to interface along their straight edges and causing core 1320 to interact with sheath 1315 in a manner that preserves the shape that guidewire 1300 had prior to stiffening.

FIG. 13E depicts three cylindrical fibers (comprising core 1320) within interior 1310. When guidewire 1300 is activated, the fibers assume a pattern of tangency that interacts with sheath 1315 in a manner that preserves the shape that guidewire 1300 had prior to stiffening. Other co-axial arrangements of fibers that cause the core and sheath to interact in a manner that preserves the guidewire's shape prior to stiffening are possible and within the scope of this invention.

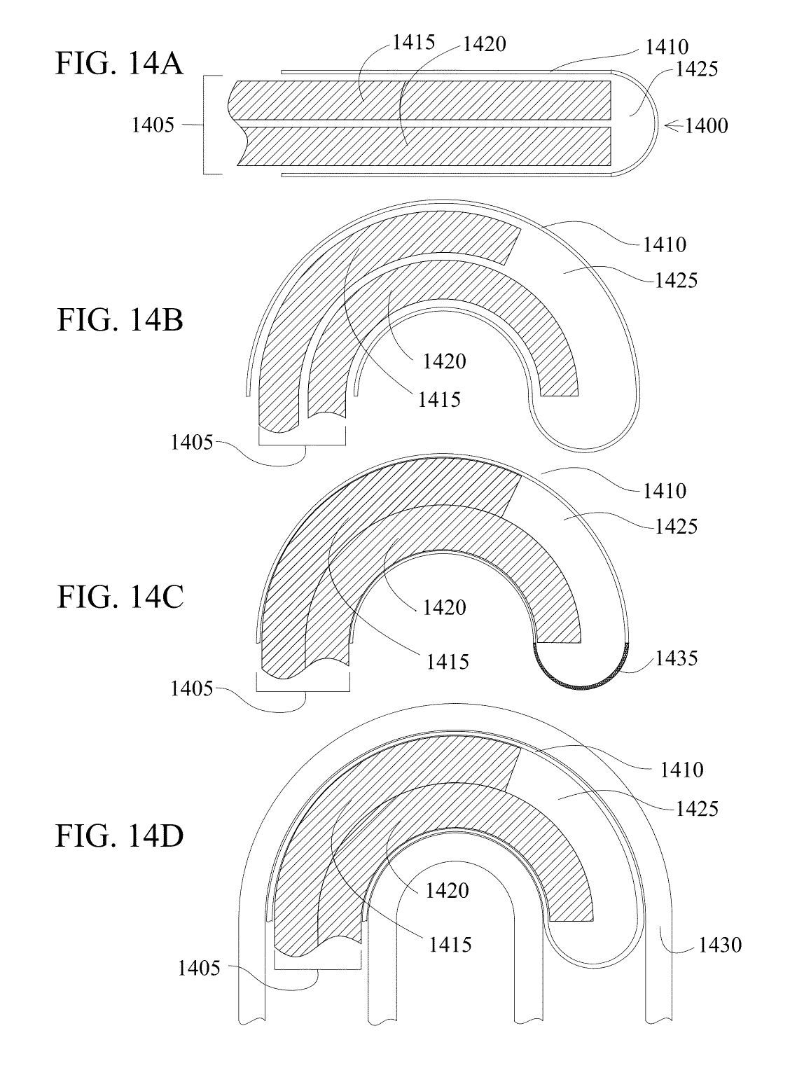

FIGS. 14A, 14B, 14C, and 14D depict another embodiment in accordance with the present invention. Guidewire 1400 includes core 1405 and sheath 1410. Core 1405 includes fiber 1415 and fiber 1420. FIG. 14A shows guidewire 1400 in a flexible state, where sheath 1410 is loosely coupled to core 1405. In this state, fiber 1415 may slide independently of fiber 1420, allowing guidewire 1400 to be bent, as shown in FIG. 14B. When guidewire 1400 is shaped in a desired manner, for instance in FIG. 14B, guidewire 1400 may be activated, causing core 1405 to couple more fixedly to sheath 1410 as shown in FIG. 14C. This activation may be achieved through various manners, including, for instance, a pressure differential, electroactive polymers embedded in sheath 1410 or core 1405, etc. (As an example, sheath 1410 may comprise an impermeable closed tube that is coupled to a pump, wherein the pump may be activated to extract fluid from interior 1425 causing sheath 1410 to retract against core 1405.) In the activated state depicted in FIG. 14C, fiber 1415 is unable to slide against fiber 1420 as independently as in the non-activated state depicted in FIG. 14B. This causes guidewire 1400 to rigidly maintain the desired shape that had been achieved prior to activation. It is contemplated that the fibers may be advantageously coated to achieve the desired effect, or constructed from one or more materials with adhesive properties, restricting movement of the fibers during the guidewire's activated stages. (Examples could include, for instance, acrylics, butyl rubber, ethylene-vinyl acetate, natural rubber, nitriles, silicone rubbers, and vinyl ethers.) Other coatings may be used to similar effect, and could be activated, for instance, via electrical charge. As a further example, the fibers may also be textured to promote mechanical adhesion during activation. Sheath 1410 may also include hard tip 1435, as shown in FIG. 14C, to prevent distortion of the tip during activation. Once guidewire 1400 has been activated into a more rigid state, overlying elements may be advantageously passed over guidewire 1400 with less possibility that guidewire 1400 will assume a shape other than that achieved prior to activation. For instance, FIG. 14D depicts element 1430 passing over guidewire 1400 in its activated state, having a desired shape. Guidewire 1400 may be deactivated (for instance be releasing the pressure differential), allowing guidewire 1400 to revert to its non-active, more flexible state.

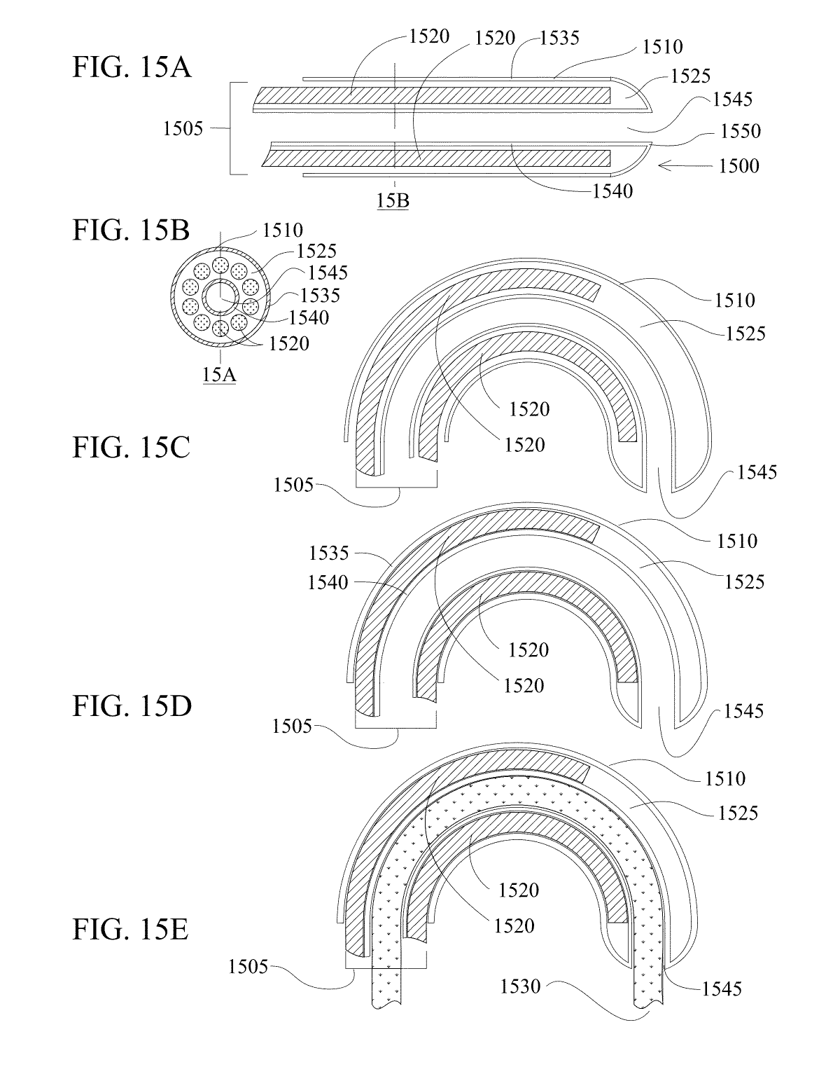

FIGS. 15A, 15B, 15C, 15D and 15E depict yet another embodiment in accordance with the present invention. Guidewire 1500 includes core 1505 and sheath 1510. Sheath 1510 includes outer-wall 1535 and inner-wall 1540, which are confluent at tip 1550 and together define interior 1525. Interior 1525 contains fiber bundle 1520 which is configured to maintain the shape of guidewire 1500. This double wall configuration creates central channel 1545 through which other devices may be passed. FIG. 15A shows guidewire 1500 in a flexible state, where sheath 1510 is loosely coupled to fiber bundle 1520 within interior 1525. FIG. 15B depicts a transverse cross-section of guidewire 1500 in an unactivated state, showing fiber bundle 1520 housed within interior 1525. In this state, the fibers within fiber bundle 1520 may slide independently, allowing guidewire 1500 to be advantageously shaped, as shown in FIG. 15C. When guidewire 1500 is shaped in a desired manner, for instance in FIG. 15D, guidewire 1500 may be activated, causing inner-wall 1540 and outer-wall 1535 to couple more fixedly to fiber bundle 1520. In the activated state depicted in FIG. 15D, the fibers within fiber bundle 1520 are unable to slide against each other and the surfaces of the inner-wall 1540 and outer-wall 1535, as independently as in the non-activated state depicted in FIG. 15C. This causes guidewire 1500 to rigidly maintain the desired shape that had been achieved prior to activation. Once guidewire 1500 has been activated into a more rigid state, elements may be advantageously passed thorough central channel 1545 with less possibility that guidewire 1500 will assume a shape other than that achieved prior to activation. For instance, FIG. 15E depicts element 1530 passing through central channel 1545. Guidewire 1500 may be deactivated (for instance by releasing the pressure differential), allowing guidewire 1500 to revert to its non-active, more flexible state.

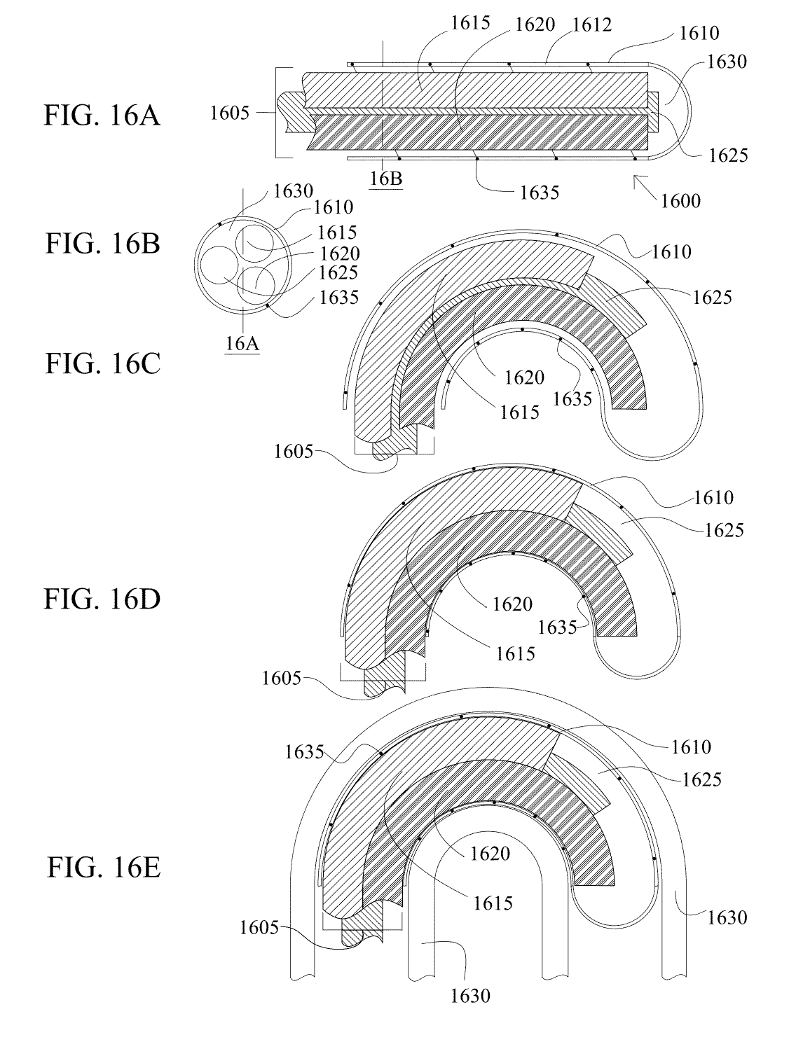

FIGS. 16A, 16B, 16C, 16D and 16E depict yet another embodiment in accordance with the present invention. Guidewire 1600 includes core 1605 and sheath 1610. Core 1605 includes fiber 1615, fiber 1620, and fiber 1625. Sheath 1610 includes electroactive polymer 1612. FIG. 16A depicts a cross-sectional view of this three-fiber arrangement within interior 1630. FIG. 16B shows guidewire 1600 in a flexible, non-activated state, where sheath 1610 is loosely coupled to core 1605. In this state, fiber 1615 may slide independently of fibers 1620 and 1625, allowing guidewire 1600 to be shaped, as shown in FIG. 16C. It is contemplated that interior 1630 may be pressurized or filled with fluid to account for environmental pressure. For instance, if guidewire 1600 is being used in a human vessel, interior 1630 may be pressurized such that sheath 1610 is appropriately loosely coupled to core 1605 to maintain a non-activated state (for instance, pressurized to approximate the environmental pressure inside a cavity). When guidewire 1600 is shaped in a desired manner, for instance in FIG. 16C, guidewire 1600 may be activated, causing core 1605 to couple more fixedly to sheath 1610 as shown in FIG. 16D. In this instance, guidewire 1600 is activated via electricity, causing electroactive polymer 1612 to flex, and retracting sheath 1610 against core 1605. In the activated state depicted in FIG. 16D, fiber 1615 is unable to slide against fiber 1620 and fiber 1625 as independently as in the non-activated state depicted in FIG. 16C. This causes guidewire 1600 to rigidly maintain the desired shape that had been achieved prior to activation. Once guidewire 1600 has been activated to a more rigid state, elements may be advantageously passed over guidewire 1600 with less possibility that guidewire 1600 will assume a shape other than that achieved prior to activation. For instance, FIG. 16E depicts element 1630 passing over guidewire 1600. Guidewire 1600 may be deactivated (for instance by releasing the pressure differential), allowing guidewire 1600 to revert to its non-active, more flexible state.

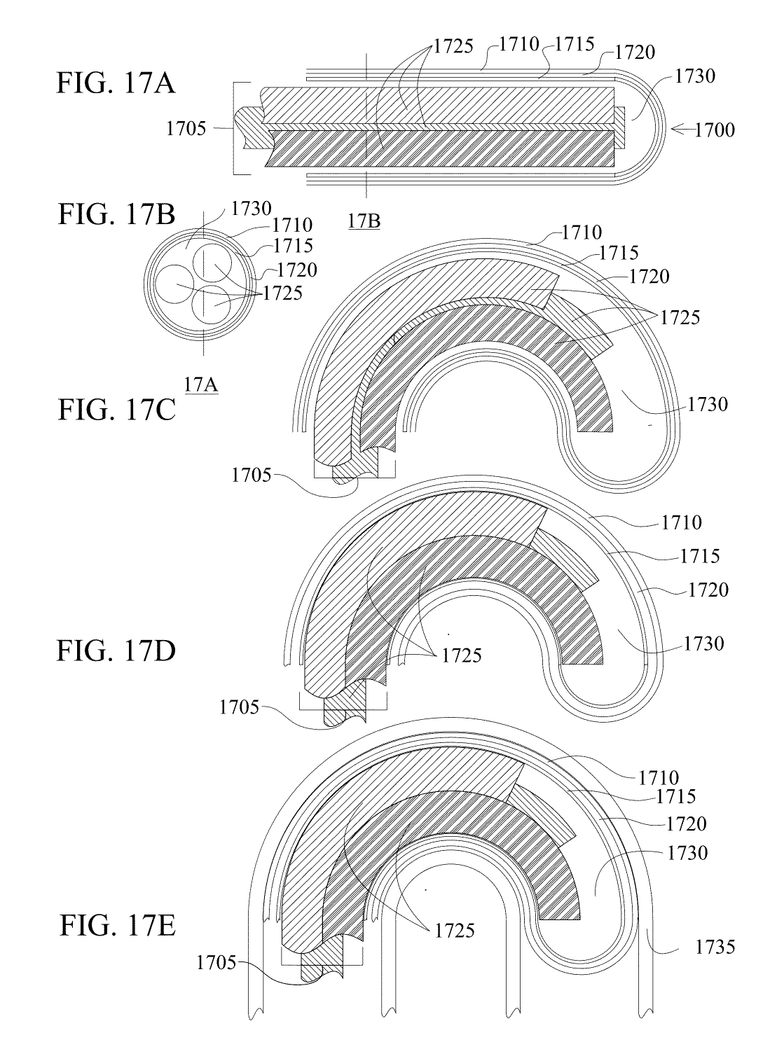

FIGS. 17A, 17B, 17C, 17D, and 17E depict another embodiment in accordance with the present invention. Guidewire 1700 includes core 1705 and a sheath comprising outer-wall 1710, inner-wall 1715 and intermediate-interior 1720. Outer-wall 1710 comprises a stiff and impermeable material whereas inner-wall 1715 comprises a flexible and impermeable material. Core 1705 includes fiber bundle 1725 comprising three parallel fibers within interior 1730. A longitudinal cross-sectional view of guidewire 1700 is depicted in FIG. 17A. FIG. 17B is a transverse cross-sectional view of guidewire 1700 in an unactivated state. In this state, intermediate-interior 1720 maintains a pressure differential relative to interior 1730 such that inner-wall 1715 is loosely coupled to core 1705 and the fibers may slide independently of one another and the sheath, allowing guidewire 1700 to be shaped, as shown in FIG. 17C. Guidewire 1700 may be stiffened by increasing the pressure in intermediate-interior 1720 causing the flexible inner-wall 1715 to retract on core 1705. In the activated state depicted in FIG. 17D, the fibers are unable to slide against one another as in the non-activated state depicted in FIG. 17C. This causes guidewire 1700 to rigidly maintain the desired shape that had been achieved prior to activation. Once guidewire 1700 has been activated into a more rigid state, elements may be advantageously passed over guidewire 1700 with less possibility that guidewire 1700 will assume a shape other than that achieved prior to activation. For instance, FIG. 17E depicts element 1735 passing over guidewire 1700. Guidewire 1700 may be deactivated (for instance by releasing the pressure differential), allowing guidewire 1700 to revert to its more flexible state.

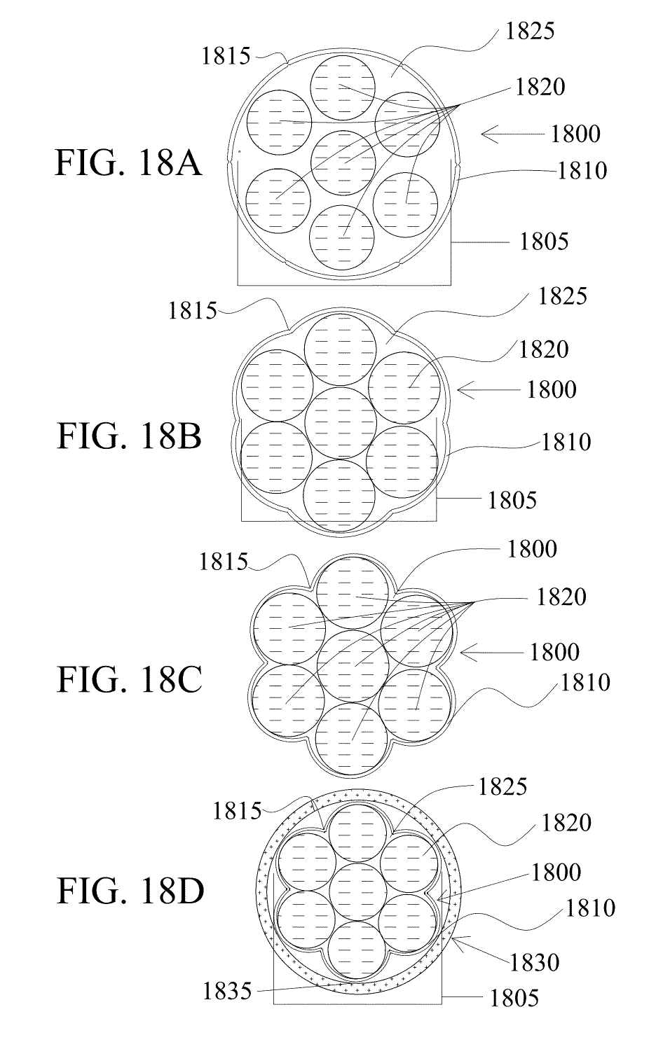

FIGS. 18A, 18B, 18C and 18D depict transverse cross sections of another embodiment in accordance with the present invention. Guidewire 1800 includes sheath 1810 and core 1805, the core comprising fiber bundle 1820 and housed within interior 1825. The fibers within fiber bundle 1820 are configured within interior 1825 to maintain the shape of guidewire 1800. Sheath 1810 has multiple zones of deformation, including zone 1815, which may result from weakness, flexibility, scoring or creasing in sheath 1810 and which may be aligned advantageously with core 1805 and fiber bundle 1820. FIG. 18A shows guidewire 1800 in an unactivated, flexible state, wherein sheath 1810 is loosely coupled to fiber bundle 1820. FIG. 18B depicts a cross section of guidewire 1800 in a state of activation that may be caused by a pressure differential between the environment and interior 1825. Activating guidewire 1800 causes sheath 1810 to retract upon core 1805 and causes folding to occur at various zones of deformation along sheath 1810 including zone 1815. FIG. 18C depicts guidewire 1800 in an advanced state of activation caused by increasing the pressure differential. In this state, sheath 1810 retracts more forcibly against core 1805 and conforms more fully to the shape of fiber bundle 1820. In this embodiment, various zones of deformation along sheath 1810 (including zone 1815) facilitate the controlled retraction of sheath 1810 while decreasing contact between sheath 1810 and external element 1830 which may pass over guidewire 1800 as depicted in FIG. 18D. Controlled folding prevents irregular folds and results in advantageous passage of external element 1830. Guidewire 1800 may be deactivated (for instance by releasing or reversing the pressure differential), allowing guidewire 1800 to revert to a more flexible state.

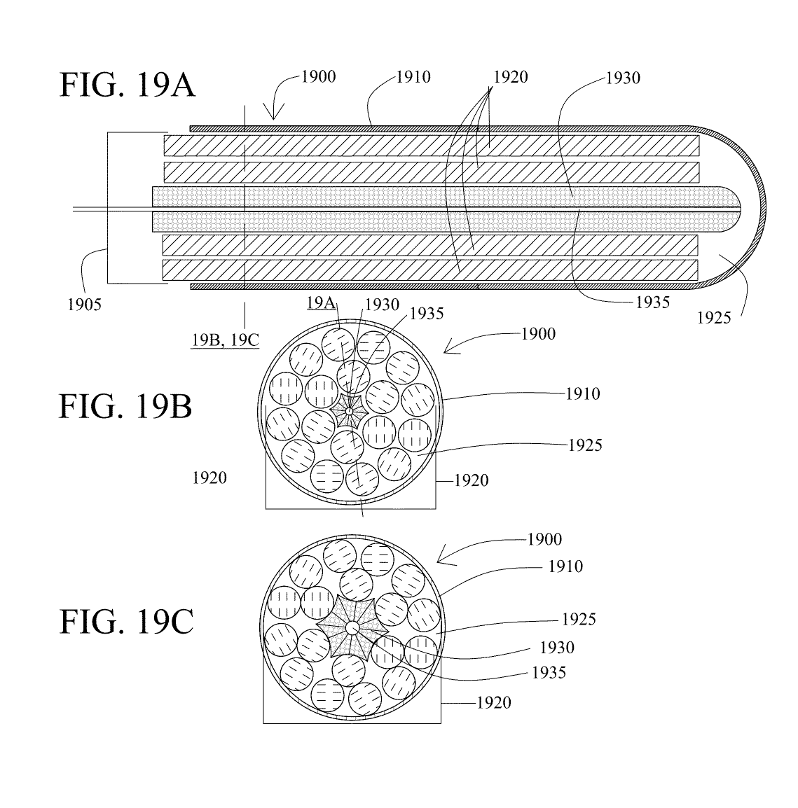

FIGS. 19A, 19B, and 19C depict another embodiment in accordance with the present invention. Guidewire 1900 includes sheath 1910 disposed about core 1905, the core being housed within interior 1925. Core 1905 comprises fiber bundle 1920 and tube 1930. Tube 1930 includes channel 1935, thorough which fluid or gas may be transmitted to tube 1930. FIG. 19A depicts guidewire 1900 in an unactivated, flexible state. Sheath 1910 comprises a rigid but flexible material and is loosely coupled to fiber bundle 1920. FIG. 19B depicts a transverse cross-section of guidewire 1900 showing fiber bundle 1920 disposed about tube 1930. In the unactivated state depicted in FIG. 19B, interior 1925 is sufficiently large relative to fiber bundle 1920 such that the fibers may slide independently of one another allowing guidewire 1900 to be shaped or bent with relative ease. When guidewire 1900 is shaped in a desired manner, it may be stiffened by transmitting fluid or gas through channel 1935 causing tube 1930 to inflate and expand against fiber bundle 1920 and causing core 1905 to couple fixedly to sheath 1910 as depicted in FIG. 19C. In this activated state, the fibers are unable to slide against one another causing guidewire 1900 to rigidly maintain the desired shape that had been achieved prior to activation. Guidewire 1900 may be deactivated by deflating tube 1930 allowing guidewire 1900 to revert to its more flexible state.

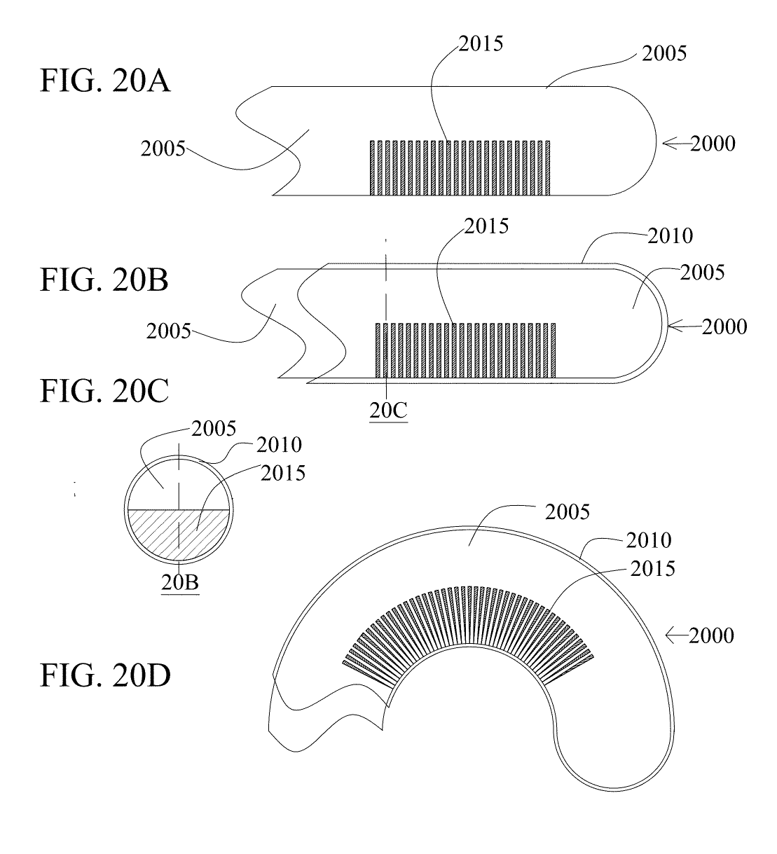

Turing now to FIG. 20A, a partial perspective view of another embodiment is depicted. FIG. 20A shows guidewire 2000's solid, internal core 2005 (sheath 2010 is not depicted in FIG. 20A). Core 2005 includes region 2015, which comprises a cutout pattern where material has been removed from core 2005. This adaptation allows region 2015 to deform into a predefined shape that may be advantageous. FIG. 20B shows guidewire 2000 with the addition of sheath 2010, which is loosely coupled to core 2005. Sheath 2010 is depicted as transparent to facilitate the explanation. FIG. 20C shows a transverse cross-sectional view of guidewire 2000, showing that the cutout pattern of region 2015 extends approximately halfway through the cross-section of core 2005. FIG. 20D shows guidewire 2000 in a state of activation, wherein sheath 2010 (transparent to aid explanation) is coupled more tightly to core 2005. For instance, guidewire 2000 may have been coupled to a pump that removed fluid from the interior of sheath 2010, tightening sheath 2010 to core 2005, causing region 2015 to deform into a predefined curve pattern. To facilitate the extraction of fluid by a pump, core 2005 may comprise a material permeable to the fluid (such as a Styrofoam-type material) or may contain channels or internal tubes which connect the pump to region 2015.

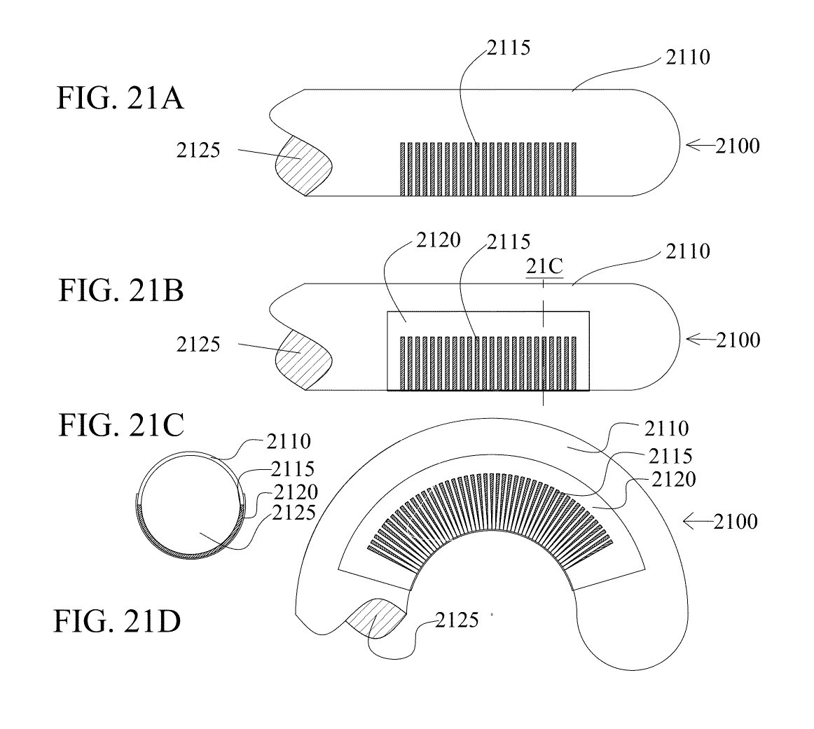

FIGS. 21A, 21B, 21C and 21D depict another embodiment in accordance with the present invention. Guidewire 2100 comprises tube 2110, which includes region 2115. Tube 2110 defines interior 2125. Region 2115 comprises an area in which portions of tube 2110 have been removed creating a reduction in thickness and strength and exposing interior 2125 to the exterior environment as depicted in FIG. 21A. FIG. 21B depicts guidewire 2100 further having cover 2120 (depicted as transparent for explanation), which is coupled to tube 2110 about region 2115. Cover 2120 is a flexible material, and separates interior 2125 from the external environment, allowing for a pressure differential to be maintained. FIG. 21C shows a cross-section of guidewire 2100, illustrating that cover 2120 in this instance is greater in size than region 2115 creating a complete barrier between interior 2125 and the exterior environment. FIG. 21D depicts guidewire 2100 in a state of activation, wherein a negative pressure has been applied to interior 2125, causing tube 2110 to deform about region 2115 to a predetermined shape. In this case, tube 2110 comprises an elastic material, such that guidewire 2100 may be returned to the unactivated state depicted in FIG. 21B upon release of the pressure differential.

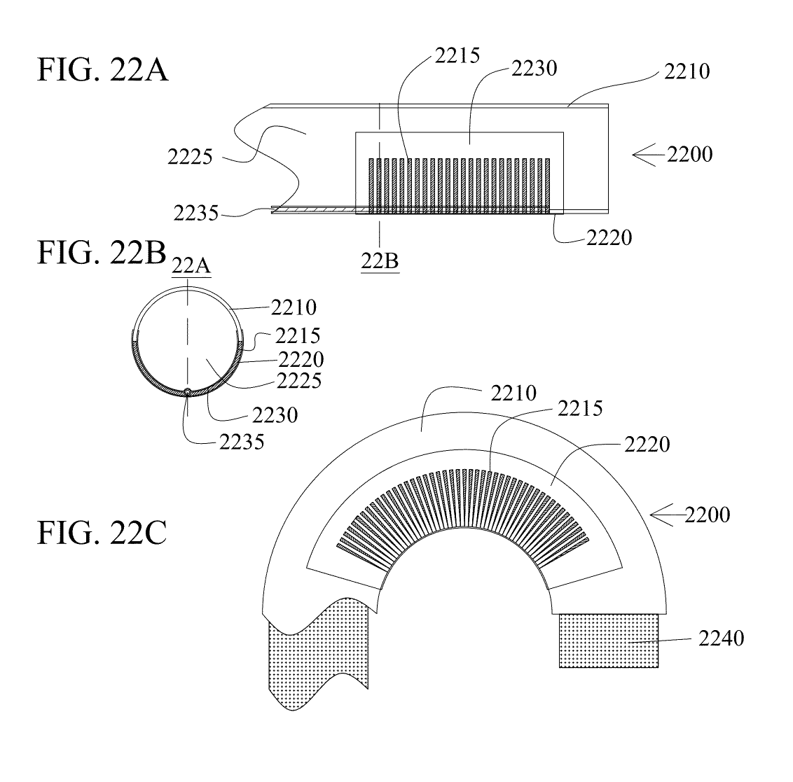

FIG. 22A is a sectional perspective view of another embodiment. Guidewire 2200 includes tube 2210, which defines interior 2225. Tube 2210 includes region 2215, which includes a cutout pattern. Cover 2230 comprises a flexible material, and is arranged over region 2215 on the inside of tube 2210. Cover 2220 comprises a flexible material, and is arranged over region 2215 on the outside of tube 2210. Together, cover 2220 and cover 2230 isolate region 2215 from interior 2225 and the environment. Guidewire 2200 further includes channel 2235, which is coupled to region 2215, allowing for the creation of a pressure differential between region 2215 and the environment. FIG. 22B depicts a cross-section of guidewire 2200, illustrating that channel 2235 passes along the wall of tube 2210. FIG. 22C illustrates guidewire 2200 in a state of activation, wherein a pressure differential has been applied to channel 2235 and, because channel 2235 is coupled to region 2215, the differential has been distributed to region 2215, causing region 2215 to deform to a defined shape. Guidewire 2200 maintains its shape while element 2240 passes through interior 2225 as depicted.

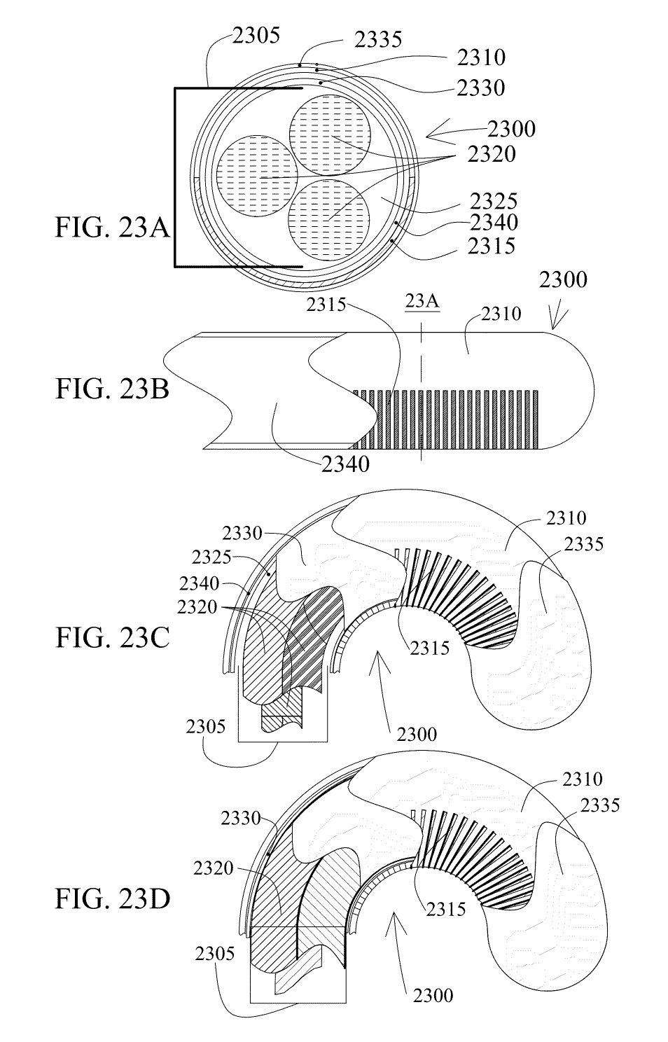

FIGS. 23A, 23B, 23C and 23D depict another embodiment in various states of activation. Guidewire 2300 comprises exterior sheath 2335, intermediate sheath 2310 and interior sheath 2330. Interior sheath 2330 defines inner-interior 2325 and is disposed about core 2305, the core comprising fiber bundle 2320. Interior sheath 2330 and exterior sheath 2335 comprise impermeable material. Intermediate sheath 2310 is coupled to exterior sheath 2335. FIG. 23A shows a transverse cross-sectional view of guidewire 2300. Interior sheath 2330 separates inner-interior 2325 from intermediate-interior 2340 allowing a pressure differential to be maintained across interior-sheath 2330. Exterior sheath 2335 separates intermediate-interior 2340 from the environment allowing a pressure differential to be maintained across exterior-sheath 2335. For example, inner-interior 2325 may assume a negative pressure differential relative to intermediate-interior 2340 causing interior-sheath to retract upon core 2305 stiffening guidewire 2300 into the shape it had assume prior to activation (“core/sheath activation”). FIG. 23B is a partial perspective view of guidewire 2300 depicting intermediate sheath 2310, which includes region 2315 comprising a series of cutouts. Guidewire 2300 may be activated by creating a negative pressure differential in intermediate-interior 2340 relative to the environment such that region 2315 deforms into a predefined shape as illustrated in FIG. 23C (“regional activation”). Once regional activation is achieved, other portions along the length of guidewire 2300 may be manipulated to shape the guidewire in a desired way before guidewire 2300 is stiffened through core/sheath activation as depicted in FIG. 23D. Thus, as can be seen, guidewire 2300 may be selectively deformed about region 2315 (via “regional activation”) independently of the stiffening of guidewire 2300 (via “core/sheath activation”).

As can be understood, embodiments of the present invention provide guidewires having variable rigidity. The present invention has been described in relation to particular embodiments, which are intended in all respects to be illustrative rather than restrictive. Alternative embodiments will become apparent to those of ordinary skill in the art to which the present invention pertains without departing from its scope, including additional apparatuses, methods, and systems. Further, while embodiments have sometimes been described in relation to the surgical field, other uses will be apparent, such as whenever guidewires having variable rigidity may be useful. It should be noted that apparatuses, methods, and systems in accordance with embodiments may also be described as devices having components configured to implement such methods, or as computer-storage media or the like having instructions for causing devices to perform such methods. Likewise, systems in accordance with embodiments may be described according to the methods they perform.

For instance, embodiments include guidewires having single or multiple cores, arranged axially, radially, circumferentially, or in any configuration necessary for the function of the device. Further, the core or cores may be tube-like and allow for the passage of devices (such as catheter, probe, needle, balloon catheter, stent, etc.) through the central portion of the core. In such an embodiment, the guidewire may be stiffened by tightening the core and sheath together, while maintaining a passage inside the core allowing for the axial transport of devices. It is contemplated that the stiffening may allow the sheath and core to tighten to the device within the core. Embodiments include guidewires having cores may be open at one or both ends, which may function to assist in the control of or contain materials that control variable stiffening, and may allow the passage of devices through or beyond the embodiment to access the vessel restriction or anatomic site. Access to these cores may be open, have controlled access through valves or other mechanisms, and may have methods for attaching external devices.

From the foregoing, it will be seen that this invention is one well adapted to attain all the ends and objects set forth above, together with other advantages which are obvious and inherent to invention. It will be understood that certain features and subcombinations are of utility and may be employed without reference to other features and subcombinations. This is contemplated by and is within the scope of the claims.

1. A guidewire comprising:
a core, the core comprising a first fiber and a second fiber, the first fiber being adjacent to the second fiber;
a sheath disposed about the core; and
a means for coupling the first fiber to the second fiber.

2. The guidewire of claim 1, wherein the guidewire is stiffened when the first fiber is coupled to the second fiber.

3. The guidewire of claim 2, wherein the sheath is configured to retract upon the core causing the first fiber and second fiber to couple.

4. The guidewire of claim 2, wherein the sheath comprises electroactive polymers configured to activate upon stimulation, causing the first fiber and second fiber to couple.

5. The guidewire of claim 2, wherein the guidewire is coupled to a pump, the pump configured to create a pressure differential across the sheath, causing the first fiber and the second fiber to couple.

6. The guidewire of claim 2, wherein the core is configured to expand upon the sheath, causing the first fiber and the sheath to couple.

7. The guidewire of claim 2, wherein the core further comprises a coil, the coil being configured to expand causing the first fiber and the sheath to couple.

8. The guidewire of claim 2, wherein the core further comprises a balloon, the balloon being configured to expand, causing the first fiber and the second fiber to couple.

9. The guidewire of claim 2, wherein the core comprises electroactive polymers configured to activate, causing the first fiber and the second fiber to couple.

10. The guidewire of claim 1, wherein the first fiber is configured to increase in adherence to the second fiber upon activation of the guidewire.

11. The guidewire of claim 1, wherein the first fiber is textured to promote coupling during activation of the guidewire.

12. The guidewire of claim 1, wherein the first fiber is coated with selectively adhesive compounds that promote coupling during activation of the guidewire.

13. A guidewire comprising:
a core, the core comprising a plurality of fibers arranged in a first prescribed pattern;
a sheath disposed about the core; and
the core and the sheath adapted to be tightened together.

14. The guidewire of claim 13, wherein upon activation, the fibers assume a second prescribed pattern.

15. The guidewire of claim 14, wherein the second prescribed pattern minimizes cross-sectional distortion of the guidewire.

16. The guidewire of claim 14, wherein the cross-sectional area occupied by the fibers arranged in the second prescribed pattern fits within the cross-sectional area occupied by the fibers arranged in the first prescribed pattern.

17. A guidewire comprising:
a core;
a sheath, comprising a first section, a second section, and a joining section coupling the first section and the second section;
the joining section being deformable relative to the first section and the second section; and wherein
the sheath is adapted to tighten to the core.

18. The guidewire of claim 17, wherein the joining section is adapted to assume a first shape when the sheath is not tightened to the core, and a second shape when the sheath is tightened to the core.

19. The guidewire of claim 17, wherein the joining section comprises flexible material that buckles upon activation.

20. The guidewire of claim 17, wherein the joining section comprises creases that fold upon activation.
